(19) United States
(12) Patent Application Publication
(10) Pub. No.: US 2026/0363624 A1
(43) Pub. Date: Nov. 19, 2026

(54) Braking Mechanism and Medical Table

(71) Applicant: Siemens Shanghai Medical Equipment Ltd., Shanghai (CN)

(72) Inventor: Ji Zhao Wang, Shanghai (CN)

(73) Assignee: Siemens Shanghai Medical Equipment Ltd., Shanghai (CN)

(21) Appl. No.: 19/132,270

(22) PCT Filed: Oct. 25, 2023
(86) PCT No.: PCT/CN2023/126563

§ 371 Date: May 22, 2025

(30) Foreign Application Priority Data
Nov. 23, 2022 (CN) 202223120399.6

(51) Int. Cl. A61B 5/00

(52) U.S. Cl. CPC A61B 5/704

(57) ABSTRACT

The present application provides a braking mechanism, which includes a first support, a first magnet, and a driving unit. The first support is configured to connect a first object. The first magnet is movably connected to the first support along a first straight line. The first magnet is selected from an electromagnet and an electrically-controlled permanent magnet. The driving unit is capable of driving the first magnet to move along the first straight line relative to the first support to enable the first magnet to reciprocate between a release position and a braking position. The first magnet in the braking position is configured to contact and magnetically attract a second object movable relative to the first object. The braking mechanism is conducive to effectively reducing noise generated during braking. In addition, the present application further provides a medical table including the braking mechanism.

### CROSS-REFERENCE TO RELATED APPLICATIONS

The present application is a U.S. national stage entry of PCT application no. PCT/CN2023/126563, filed Oct. 25, 2023, which claims priority to and the benefit of China patent application no. CN 202223120399.6, filed on Nov. 23, 2022, the contents of each of which are incorporated herein by reference in their entireties.

### TECHNICAL FIELD

The present application relates to a braking mechanism and a medical table including the braking mechanism.

### BACKGROUND

In the medical field, the table board of the medical table is typically arranged to be able to move relative to the table frame, so as to facilitate examination and treatment. An electromagnetic braking mechanism is often used for braking between the table board and the table frame. In the existing electromagnetic braking mechanism, when the electromagnet is energized, due to the effect of the magnetic force, the electromagnet is attracted by the table board and quickly moves until it abuts against the table board. Since the electromagnet moves at a very high speed, the collision noise when the electromagnet contacts the table board is very loud.

### SUMMARY

An objective of the present application is to provide a braking mechanism, which is conducive to reducing noise generated during braking.

Another objective of the present application is to provide a medical table, whose braking mechanism is conducive to reducing noise generated during braking.

The present application provides a braking mechanism, which includes a first support, a first magnet and a driving unit. The first support is configured to connect a first object. The first magnet is movably connected to the first support along a first straight line. The first magnet is selected from an electromagnet and an electrically-controlled permanent magnet. The driving unit is capable of driving the first magnet to move along the first straight line relative to the first support to enable the first magnet to reciprocate between a release position and a braking position. The first magnet in the braking position is configured to contact and magnetically attract a second object movable relative to the first object.

During braking of the braking mechanism, the driving unit firstly drives the first magnet to the braking position to contact the second object, and then controls the first magnet to generate a magnetic field to attract the second object. By properly arranging the driving unit, the noise generated when the first magnet comes into contact with the second object can be reduced to an acceptable range. The arrangement of the driving unit does not affect the magnitude of the magnetic attraction of the first magnet to the second object. Thereby, on the basis of ensuring the braking effect, the braking mechanism is conducive to effectively reducing the noise generated during braking.

In another schematic embodiment of the braking mechanism, the driving unit includes a first elastic member and a driving assembly. The first elastic member is capable of applying an elastic force to drive the first magnet to move between the release position and the braking position along a first direction parallel to the first straight line relative to the first support. The driving assembly includes an iron core and a second magnet. The iron core movably runs through the second magnet along the first straight line. The second magnet is selected from an electromagnet and an electrically-controlled permanent magnet. A magnetic field generated by the second magnet is capable of driving the iron core to move along the first straight line relative to the second magnet. One of the iron core and the second magnet is fixedly arranged relative to the first support. The other one of the iron core and the second magnet is fixedly arranged relative to the first magnet to drive the iron core to move by the second magnet to drive the first magnet to move between the release position and the braking position along a direction opposite to the first direction. This structure is simple and low in cost.

In still another schematic embodiment of the braking mechanism, the braking mechanism further includes a second support. The second support is movably connected to the first support along the first straight line. The first magnet and the second magnet are fixedly arranged on the second support. The iron core is fixedly arranged on the first support. In this way, the assembly is facilitated.

In yet another schematic embodiment of the braking mechanism, the first support has a first flat plate portion. The second support has a second flat plate portion. The first flat plate portion and the second flat plate portion are both arranged perpendicular to the first direction and overlapped with each other along the first direction. The first magnet and the second magnet are arranged on a side of the second flat plate portion facing away from the first flat plate portion. In this way, the structure is more compact and the space can be saved.

In yet another schematic embodiment of the braking mechanism, one of the first flat plate portion and the second flat plate portion is provided with a slide hole penetrating along the first direction, and the other one of the first flat plate portion and the second flat plate portion is provided with a slide bar. The slide bar slidably runs through the slide hole along the first direction. In this way, the structure is simple and stable.

In yet another schematic embodiment of the braking mechanism, the first direction is a direction in which the first magnet moves from the braking position to the release position. The second flat plate portion has the slide hole. The first support has the slide bar. An end of the slide bar located on the second flat plate portion and facing away from the first flat plate portion has a boss portion. The first elastic member is a compression spring and is sleeved on the slide bar. One end of the first elastic member abuts against the second flat plate portion, the first magnet or the second magnet, and the other end abuts against the boss portion. In this way, the structure is simple and stable.

In yet another schematic embodiment of the braking mechanism, the first direction is a direction in which the first magnet moves from the release position to the braking position. The second flat plate portion has an avoiding hole. The first magnet has a first accommodating cavity opposite to the avoiding hole along the first direction. The first elastic member runs through the avoiding hole. One end of the first elastic member abuts against the first flat plate portion, and the other end extends into the first accommodating cavity and abuts against the first magnet. The structure is simple and the space can be saved.

In yet another schematic embodiment of the braking mechanism, the braking mechanism is provided with a plurality of first magnets. At least a part of the plurality of first magnets are electrically-controlled permanent magnets. The advantage of arranging the electrically-controlled permanent magnets is that the electrically-controlled permanent magnets can maintain their magnetism after an accidental power failure of the system, and thus, can be used for avoiding a braking failure after the power failure.

In yet another schematic embodiment of the braking mechanism, the braking mechanism further includes a connecting member, a second elastic member and an abutting member. The connecting member is movably connected to the first support along the first straight line. The first support is connected to the first object through the connecting member. The second elastic member is capable of applying an elastic force to drive the first support to move relative to the connecting member in a direction the same as the direction in which the first magnet moves from the release position to the braking position. The abutting member is connected to the first support to abut against the second object under the action of the elastic force of the second elastic member. When in use, even if a relative distance between the first object and the second object along the first straight line is slightly offset, the abutting member can also abut against the second object under the action of the elastic force of the second elastic member, so as to keep the relative position between the first support and the second object along the first straight line constant, thereby ensuring that the first magnet in the braking position can contact the second object.

In yet another schematic embodiment of the braking mechanism, the abutting member is a roller wheel, and the roller wheel is rotatably connected to the first support with a rotation axis perpendicular to the first straight line. In this way, mutual friction between the abutting member and the second object can be reduced.

In yet another schematic embodiment of the braking mechanism, the connecting member is a pin and runs through the first support. The second elastic member is a compression spring and is sleeved on the connecting member. This structure is simple and low in cost.

In yet another schematic embodiment of the braking mechanism, the braking mechanism is provided with a plurality of connecting members. The first support includes a first flat plate portion and a plurality of connecting portions. The first flat plate portion is in a flat plate shape perpendicular to the first straight line. Each of the connecting portions is in a flat plate shape parallel to the first flat plate portion. Each of the connecting members runs through one of the connecting portions. The first magnet, the abutting member and the connecting portions are located on a same side of the first flat plate portion. In this way, the structure is more compact and the space can be saved.

The present application provides a medical table, which includes a table frame, a table board and a braking mechanism described above. The table board is movably arranged on the table frame. The first support is connected to one of the table frame and the table board. The other one of the table frame and the table board contacts the first magnet in the braking position and is capable of being magnetically attracted by the magnetic force of the first magnet. On the basis of ensuring the braking effect, the braking mechanism of the medical table is conducive to effectively reducing the noise generated during braking.

### BRIEF DESCRIPTION OF THE DRAWINGS

The following accompanying drawings merely illustrate and explain the present application schematically, and do not limit the scope of the present application.

FIG. 1 is a three-dimensional view of a schematic embodiment of a braking mechanism.

FIG. 2 is a partial sectional view of the braking mechanism shown in FIG. 1.

FIG. 3 is a schematic exploded view of the structure shown in FIG. 2.

FIG. 4 is a schematic view of another state of the structure shown in FIG. 2.

FIG. 5 is a partial sectional view of another schematic embodiment of a braking mechanism.

FIG. 6 is a partial sectional view of still another schematic embodiment of a braking mechanism.

FIG. 7 is a schematic exploded view of the structure shown in FIG. 6.

FIG. 8 is a schematic view of another state of the structure shown in FIG. 6.

FIG. 9 is a schematic structural view of a schematic embodiment of a medical table.

### DESCRIPTION OF SYMBOLS

100 braking mechanism
10 first support
11 first flat plate portion
12 connecting portion
20 second support
21 second flat plate portion
211 avoiding hole
30 first magnet
31 first accommodating cavity
41 slide hole
42 slide bar
421 boss portion
51 first elastic member
52 driving assembly
521 iron core
522 second magnet
71 connecting member
72 second elastic member
73 abutting member
81 table frame
82 table board
91 first object
92 second object
L1 first straight lineD1 first direction

### DETAILED DESCRIPTION

For a clearer understanding of the technical features, objectives and effects of the present application, the specific embodiments of the present application will be described with reference to the accompanying drawings, in which like reference numerals refer to components having the same structure or similar structure but having the same function.

Here, “schematic” means “serving as an example, instance or illustration”, and any illustration or embodiments described herein as “schematic” should not be interpreted as a more preferred or more advantageous technical solution.

Herein, “first”, “second” and the like are not intended to indicate their importance or sequence and the like, but only used for distinguishing each other to facilitate the description of the document.

For the sake of brevity, only the parts related to the present application are schematically shown in the drawings, but they do not represent the actual structure of the product.

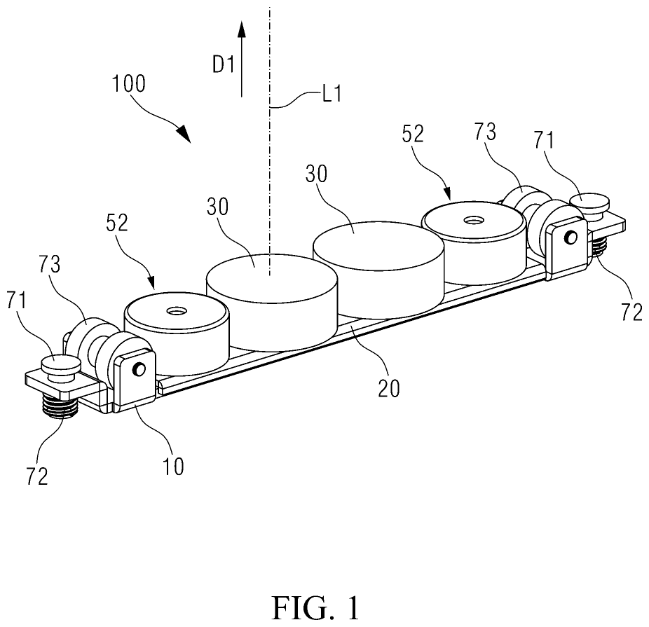

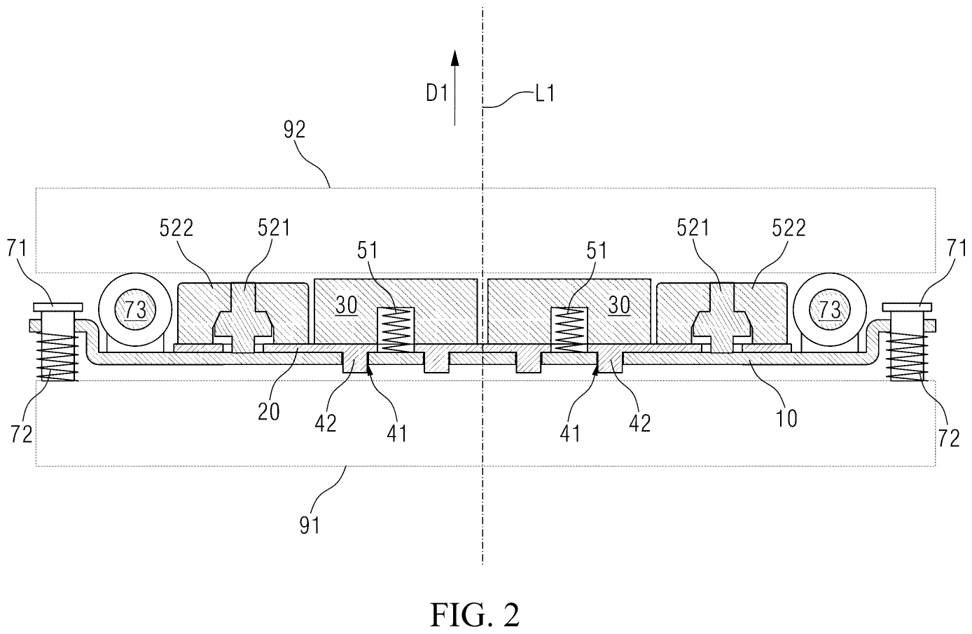

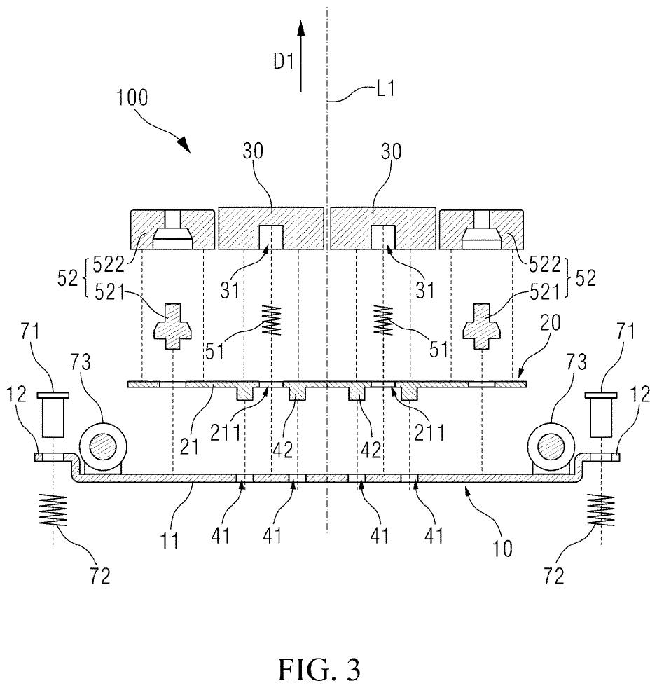

FIG. 1 is a three-dimensional view of a schematic embodiment of a braking mechanism. FIG. 2 is a partial sectional view of the braking mechanism shown in FIG. 1. FIG. 3 is a schematic exploded view of the structure shown in FIG. 2. The braking mechanism is, for example, used for braking a first object 91 and a second object 92 that are movable relative to each other in a direction, for example, perpendicular to a first straight line L1 in the figure. For example, the braking mechanism may be a braking mechanism for a table structure. In this case, the first object 91 and the second object 92 are, for example, but not limited to, a table frame and a table board movably connected to the table frame respectively. As shown in FIG. 1 to FIG. 3, a braking mechanism 100 includes a first support 10, two first magnets 30 and a driving unit.

Each of the first magnet 30 is movably connected to the first support 10 along the first straight line L1. Specifically, in this schematic embodiment, the braking mechanism 100 further includes a second support 20. The second support 20 is movably connected to the first support 10 along the first straight line L1. The first magnets 30 are fixedly arranged on the second support 20, that is, the first magnets 30 are movably connected to the first support 10 through the second support 20.

Further, as shown in FIG. 3, in this schematic embodiment, the first support 10 has a first flat plate portion 11. The second support 20 has a second flat plate portion 21. The first flat plate portion 11 and the second flat plate portion 21 are both arranged perpendicular to the first straight line L1 and overlapped with each other along a direction parallel to the first straight line L1. The first magnets 30 are arranged on a side of the second flat plate portion 21 facing away from the first flat plate portion 11. In this way, the overall structure is more compact and the space can be saved.

Further, as shown in FIG. 3, in this schematic embodiment, the first flat plate portion 11 is provided with a plurality of slide holes 41 penetrating along the first straight line L1, and the second flat plate portion 21 is provided with a plurality of slide bars 42 (only two slide bars 42 are shown in FIG. 3). Each of the slide bars 42 slidably runs through one slide hole 41 along the direction parallel to the first straight line L1. In this way, the movable connection between the first support 10 and the second support 20 is realized through the slide hole 41 and the slide bar 42. However, the present application is not limited thereto, in other schematic embodiments, it may also be that the second flat plate portion 21 is provided with the slide hole 41, and the first flat plate portion 11 is provided with the slide bar 42. The structure of the slide hole and the slide bar is simple and stable, but the present application is not limited thereto. In other schematic embodiments, the movable connection between the first support 10 and the second support 20 may also be realized through other structures.

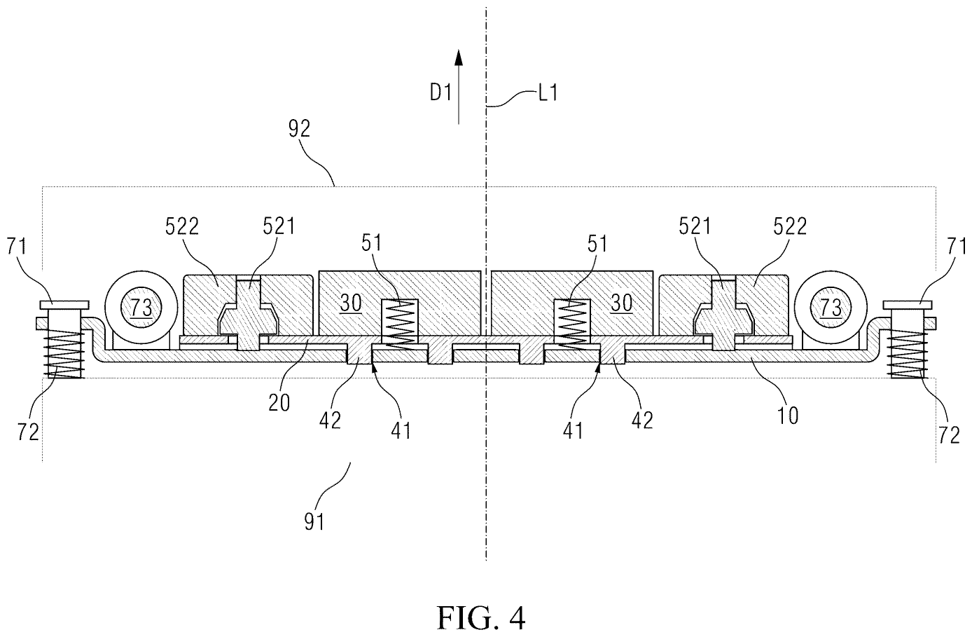

The driving unit is capable of driving the first magnet 30 to move along the first straight line L1 relative to the first support 10 to enable the first magnet 30 to reciprocate between a release position and a braking position. The first magnet 30 shown in FIG. 2 is in the release position. FIG. 4 is a schematic view of another state of the structure shown in FIG. 2, and the first magnet 30 shown in FIG. 4 is in the braking position. The first magnet 30 in the braking position is configured to contact and magnetically attract the second object 92.

Specifically, as shown in FIG. 2 and FIG. 3, in this schematic embodiment, the driving unit includes two first elastic members 51 and two driving assemblies 52. The first elastic member 51 is capable of applying an elastic force to drive the first magnet 30 to move from the release position to the braking position along a first direction D1 parallel to the first straight line L1 relative to the first support 10. As shown in FIG. 3, in this schematic embodiment, the second flat plate portion 21 has two avoiding holes 211. Each of the first magnets 30 has a first accommodating cavity 31 opposite to one of the avoiding holes 211 along the first direction D1. Each of the first elastic members 51 runs through one of the avoiding holes 211. One end of the first elastic member 51 abuts against the first flat plate portion 11, and the other end extends into the first accommodating cavity 31 and abuts against the first magnet 30. In this way, the overall structure is more compact and the space can be saved, but the present application is not limited thereto.

As shown in FIG. 2 and FIG. 3, each driving assembly 52 includes an iron core 521 and a second magnet 522. The iron core 521 movably runs through the second magnet 522 along the first straight line L1. The second magnet 522 is selected from an electromagnet and an electrically-controlled permanent magnet. A magnetic field generated by the second magnet 522 is capable of driving the iron core 521 to move along the first straight line L1 relative to the second magnet 522, based on a principle similar to that of a push-pull electromagnet. The iron core 521 is fixedly arranged on the first support 10. The second magnet 522 is fixedly arranged on the second support 20 to drive the iron core 521 to move by the second magnet 522, so as to overcome the elastic force of the first elastic member 51 and drive the first magnet 30 to move from the braking position to the release position along a direction opposite to the first direction D1. The second magnet 522 is arranged on a side of the second flat plate portion 21 facing away from the first flat plate portion 11 to save the space, but the present application is not limited thereto.

The first support 10 is configured to connect the first object 91. Specifically, as shown in FIG. 2 and FIG. 3, in this schematic embodiment, the braking mechanism 100 further includes two connecting members 71, two second elastic members 72 and two abutting members 73. The connecting member 71 is movably connected to the first support 10 along the first straight line L1. The connecting member 71 is, for example, a pin, and runs through the first support 10, but the present application is not limited thereto. When in use, as shown in FIG. 2, the connecting member 71 is fixedly connected to the first object 91, and thereby, the first support 10 is connected to the first object 91 through the connecting member 71. The second elastic member 72 is capable of applying an elastic force to drive the first support 10 to move relative to the connecting member 71 in a direction the same as the direction in which the first magnet 30 moves from the release position to the braking position. The second elastic member 72 is, for example, a compression spring, and is sleeved on the connecting member 71, but the present application is not limited thereto. The abutting member 73 is connected to the first support 10 to abut against the second object 92 under the action of the elastic force of the second elastic member 72. When in use, even if a relative distance between the first object 91 and the second object 92 along the first straight line L1 is slightly offset, the abutting member 73 can also abut against the second object 92 under the action of the elastic force of the second elastic member 72, so as to keep the relative position between the first support 10 and the second object 92 along the first straight line L1 constant, thereby ensuring that the first magnet 30 in the braking position can contact the second object 92. In other schematic embodiments, if the relative distance between the first object 91 and the second object 92 along the first straight line L1 is accurately controlled, the first support 10 may also be directly fixedly connected to the first object 91, and there is no need to provide the connecting member 71, the second elastic member 72 and the abutting member 73. In this schematic embodiment, the abutting member 73 is, for example, a roller wheel, and the roller wheel is rotatably connected to the first support 10 with a rotation axis perpendicular to the first straight line L1 and the direction in which the first object 91 and the second object 92 move relative to each other, thereby reducing the mutual friction between the abutting member and the second object.

In this schematic embodiment, the first magnet 30 is selected from an electromagnet and an electrically-controlled permanent magnet. That is, the two first magnets 30 are both electromagnets; or the two first magnets 30 are both electrically-controlled permanent magnets; or one of the first magnets 30 is an electromagnet, and the other one of the first magnets 30 is an electrically-controlled permanent magnet. The advantage of arranging the electrically-controlled permanent magnets is that the electrically-controlled permanent magnets can maintain their magnetism after an accidental power failure of the system, and thus, can be used for avoiding a braking failure after the power failure.

A schematic usage scenario of the braking mechanism is described below. Before braking, the driving unit is controlled to keep the first magnet 30 in the release position, and the first magnet 30 is controlled to make its magnetic field disappear. During braking, firstly, the driving unit is controlled to make the first magnet 30 move from the release position to the braking position to contact the second object 92, and then the first magnet 30 is controlled to generate the magnetic field to attract the second object 92. To cancel the braking, firstly, the first magnet 30 is controlled to make its magnetic field disappear, and then the driving unit is controlled to make the first magnet 30 move from the braking position to the release position.

Specifically, in this schematic embodiment, the control of the driving unit is realized by controlling the disappearance and generation of the magnetic field of the second magnet 522. In this schematic embodiment, for the electromagnet, when the electromagnet is energized, the magnetic field is generated; and when the electromagnet is deenergized, the magnetic field disappears. For the electrically-controlled permanent magnet, when the electrically-controlled permanent magnet is energized, the magnetic field disappears; and when the electrically-controlled permanent magnet is deenergized, the magnetic field is generated. In this way, the first magnet 30 and the second magnet 522 are controlled.

During braking of the braking mechanism, the driving unit firstly drives the first magnet 30 to the braking position to contact the second object 92, and then controls the first magnet 30 to generate a magnetic field to attract the second object 92. By properly arranging the driving unit, the noise generated when the first magnet 30 comes into contact with the second object 92 can be reduced to an acceptable range. The arrangement of the driving unit does not affect the magnitude of the magnetic attraction of the first magnet 30 to the second object 92. Thereby, on the basis of ensuring the braking effect, the braking mechanism is conducive to effectively reducing the noise generated during braking.

The driving unit of this schematic embodiment is simple in structure and low in cost, but the present application is not limited thereto. In other schematic embodiments, the driving unit may also be other structures, as long as it can drive the first magnet 30 to reciprocate between the release position and the braking position, for example, the driving unit may be a motor or a push-pull electromagnet.

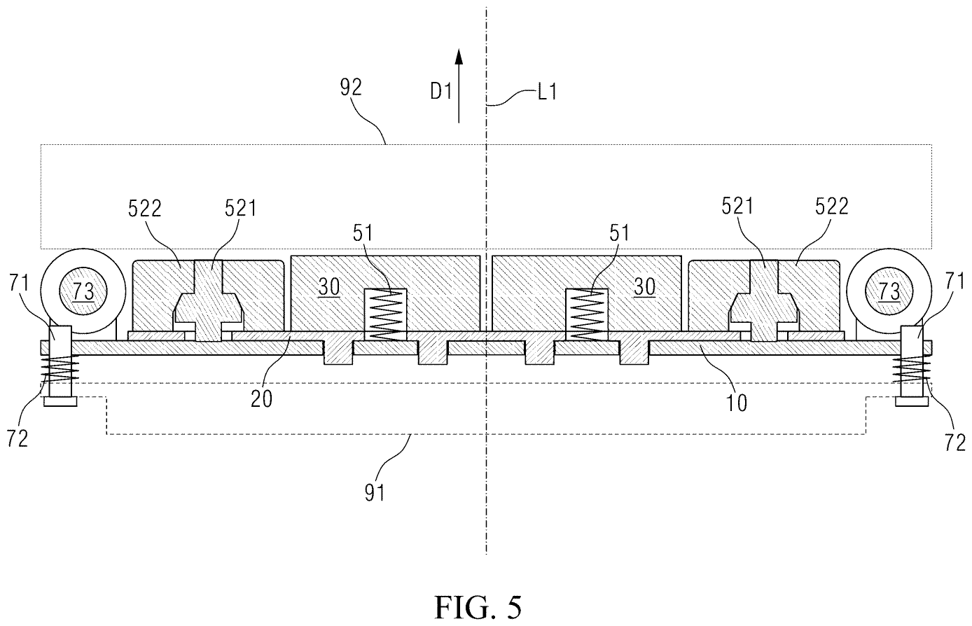

As shown in FIG. 3, in this schematic embodiment, the first support 10 further includes two connecting portions 12. Each of the connecting portions 12 is in a flat plate shape parallel to the first flat plate portion 11. Each of the connecting members 71 runs through one of the connecting portions 12. The second flat plate portion 21, the driving assembly 52, the first magnet 30, the abutting member 73 and the connecting portions 12 are located on the same side of the first flat plate portion 11. In this way, the overall structure is more compact and the space can be saved, but the present application is not limited thereto. In other schematic embodiments, as shown in FIG. 5, the first support 10 may also not be provided with the connecting portions 12, and the connecting member 71 runs through the first flat plate portion 11.

In other schematic embodiments, the numbers of the first magnet 30, the first elastic member 51, the driving assembly 52, the connecting member 71, the second elastic member 72 and the abutting member 73 may be adjusted according to needs.

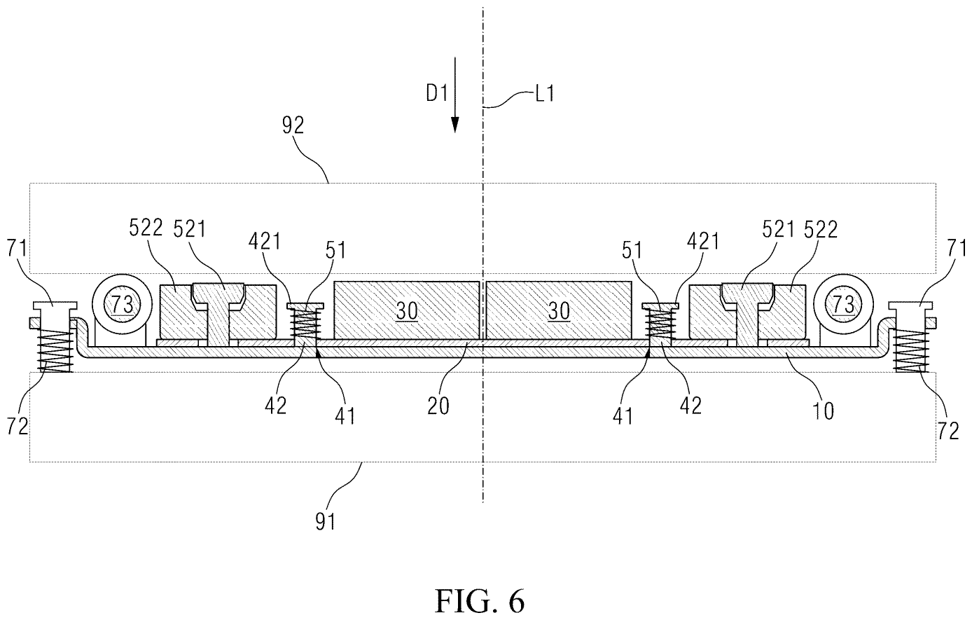

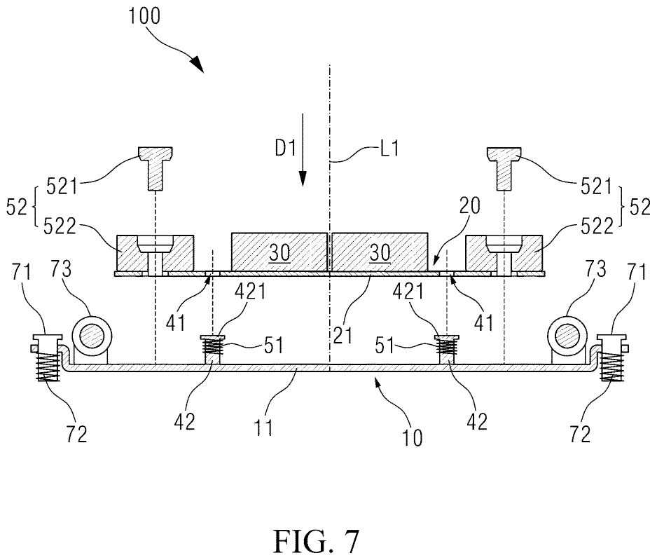

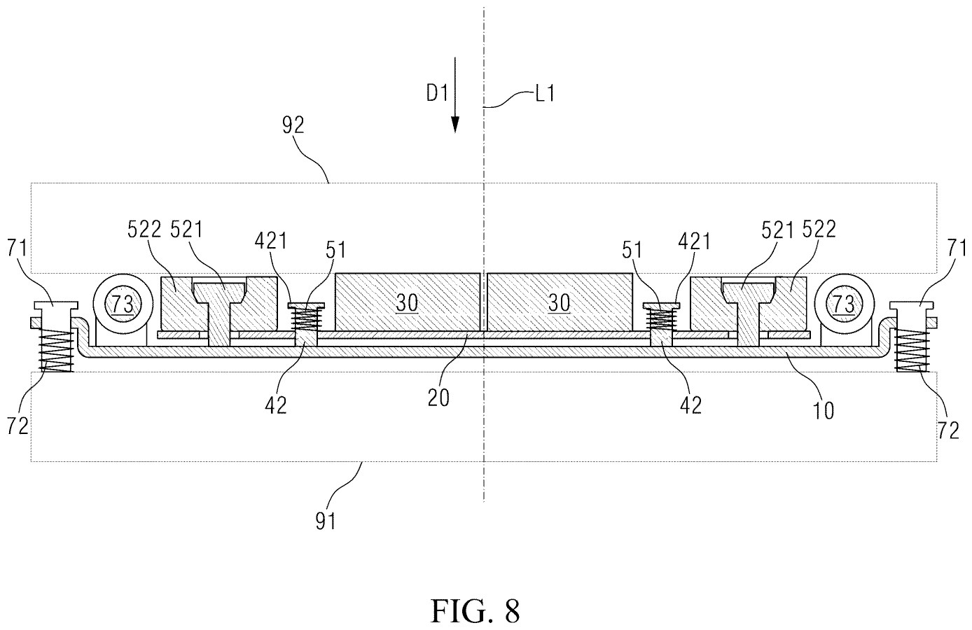

FIG. 6 is a partial sectional view of still another schematic embodiment of a braking mechanism. FIG. 7 is a schematic exploded view of the structure shown in FIG. 6. FIG. 8 is a schematic view of another state of the structure shown in FIG. 6. The parts of the braking mechanism of this schematic embodiment that are the same as or similar to the braking mechanism shown in FIG. 2 will not be repeated here, and the differences are as follows. The first magnet 30 shown in FIG. 6 is in the release position, and the first magnet 30 shown in FIG. 8 is in the braking position. As shown in FIG. 6 to FIG. 8, in this schematic embodiment, the first direction D1 is a direction in which the first magnet 30 moves from the braking position to the release position. The slide hole 41 is arranged in the second flat plate portion 21. The slide bar 42 is arranged in the first flat plate portion 11. An end of the slide bar 42 located on the second flat plate portion 21 and facing away from the first flat plate portion 11 has a boss portion 421. The first elastic member 51 is a compression spring and is sleeved on the slide bar 42. One end of the first elastic member 51 abuts against the second flat plate portion 21, and the other end abuts against the boss portion 421. In other schematic embodiments, the slide bar 42 may also be arranged to run through the first magnet 30 or the second magnet 522; and one end of the first elastic member 51 abuts against the first magnet 30 or the second magnet 522 through which the slide bar 42 runs, and the other end abuts against the boss portion 421.

In this schematic embodiment, the first magnet 30 can be driven by the elastic force of the first elastic member 51 to move from the braking position to the release position, and the magnetic force of the second magnet 522 in the driving assembly 52 applied to the iron core 521 can overcome the elastic force of the first elastic member 51, so as to drive the first magnet 30 to move from the release position to the braking position.

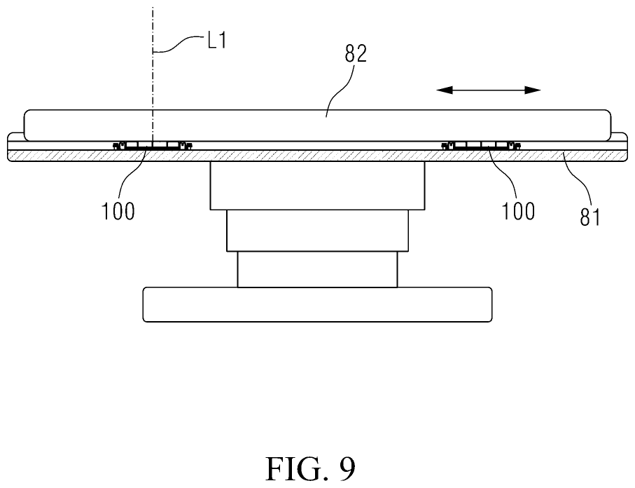

FIG. 9 is a schematic structural view of a schematic embodiment of a medical table. The medical table includes a table frame 81, a table board 82 and a plurality of the braking mechanisms 100 shown in FIG. 1, FIG. 5 or FIG. 6. The table board 82 is movably arranged on the table frame 81. The moving direction of the table board, for example, is shown as the bidirectional arrow in the figure. The moving direction is, for example, perpendicular to the first straight line L1. The first support 10 of the braking mechanism 100 is connected to the table frame 81. The table board 82 contacts the first magnet 30 in the braking position and is capable of being magnetically attracted by the magnetic force of the first magnet 30. On the basis of ensuring the braking effect, the braking mechanism of the medical table is conducive to effectively reducing the noise generated during braking.

It should be understood that although this specification is described in accordance with individual examples, not every example includes only one independent technical solution. This description manner of the specification is merely for clarity, and those skilled in the art should consider the specification as a whole. The technical solutions in the examples can also be combined as appropriate to form other embodiments that can be understood by those skilled in the art.

The series of detailed descriptions listed above are only specific descriptions of the feasible examples of the present application, and they are not intended to limit the protection scope of the present application. Any equivalent embodiment or change made without departing from the spirit of the present application, such as combination, division or repetition of features, should be included within the protection scope of the present application.

1.-13. (canceled)

14. A braking mechanism, comprising:
a first support configured to connect a first object;
a first magnet movably connected to the first support along a first straight line, the first magnet comprising one of an electromagnet or an electrically-controlled permanent magnet; and
a driving unit configured to drive the first magnet to move along the first straight line relative to the first support to enable the first magnet to reciprocate between a release position and a braking position,
wherein the first magnet, in the braking position, is configured to contact and magnetically attract a second object that is movable relative to the first object.

15. The braking mechanism according to claim 14, wherein the driving unit comprises:
a first elastic member configured to apply an elastic force to drive the first magnet to move between the release position and the braking position along a first direction that is parallel to the first straight line relative to the first support; and
a driving assembly comprising an iron core and a second magnet, and wherein:
the iron core is configured to movably run through the second magnet along the first straight line,
the second magnet comprises one of an electromagnet or an electrically-controlled permanent magnet,
a magnetic field generated by the second magnet drives the iron core to move along the first straight line relative to the second magnet, and
one of the iron core or the second magnet is fixedly arranged relative to the first support, and the other one of the iron core or the second magnet is fixedly arranged relative to the first magnet to drive the iron core to move by the second magnet and to drive the first magnet to move between the release position and the braking position along a direction opposite to the first direction.

16. The braking mechanism according to claim 15, wherein the braking mechanism further comprises a second support that is movably connected to the first support along the first straight line,
wherein the first magnet and the second magnet are fixedly arranged on the second support, and
wherein the iron core is fixedly arranged on the first support.

17. The braking mechanism according to claim 16, wherein:
the first support comprises a first flat plate portion,
the second support comprises a second flat plate portion,
the first flat plate portion and the second flat plate portion are arranged perpendicular to the first direction and overlap with one other along the first direction, and
the first magnet and the second magnet are arranged on a side of the second flat plate portion facing away from the first flat plate portion.

18. The braking mechanism according to claim 17, wherein one of the first flat plate portion or the second flat plate portion is provided with a slide hole penetrating along the first direction, and
wherein the other one of the first flat plate portion or the second flat plate portion is provided with a slide bar slidably running through the slide hole along the first direction.

19. The braking mechanism according to claim 18, wherein:
the first direction comprises a direction in which the first magnet moves from the braking position to the release position,
the second flat plate portion comprises the slide hole,
the first support comprises the slide bar, an end of the slide bar that is located on the second flat plate portion and facing away from the first flat plate portion having a boss portion,
the first elastic member comprises a compression spring and is sleeved on the slide bar,
one end of the first elastic member abuts against the second flat plate portion, the first magnet, or the second magnet, and the other end of the first elastic member abuts against the boss portion.

20. The braking mechanism according to claim 17, wherein:
the first direction comprises a direction in which the first magnet moves from the release position to the braking position,
the second flat plate portion comprises an avoiding hole,
the first magnet comprises a first accommodating cavity opposite to the avoiding hole along the first direction,
the first elastic member runs through the avoiding hole, and
one end of the first elastic member abuts against the first flat plate portion, and the other end of the first elastic member extends into the first accommodating cavity and abuts the first magnet.

21. The braking mechanism according to claim 14, wherein the first magnet is from among a plurality of first magnets, and
wherein the braking mechanism comprises the plurality of the first magnets, at least a portion of the plurality of first magnets comprising electrically-controlled permanent magnets.

22. The braking mechanism according to claim 14, wherein the braking mechanism further comprises:
a connecting member movably connected to the first support along the first straight line, the first support being connected to the first object through the connecting member;
a second elastic member configured to apply an elastic force to drive the first support to move relative to the connecting member in a direction that is the same as the direction in which the first magnet moves from the release position to the braking position; and
an abutting member connected to the first support and configured to abut against the second object under action of the elastic force of the second elastic member.

23. The braking mechanism according to claim 22, wherein the abutting member comprises a roller wheel that is rotatably connected to the first support with a rotation axis perpendicular to the first straight line.

24. The braking mechanism according to claim 22, wherein the connecting member comprises a pin and runs through the first support, and
wherein the second elastic member comprises a compression spring that is sleeved on the connecting member.

25. The braking mechanism according to claim 24, wherein the braking mechanism is provided with a plurality of the connecting members, and
wherein the first support comprises:
a first flat plate portion in a flat plate shape perpendicular to the first straight line; and
a plurality of connecting portions,
wherein each of the plurality of connecting portions are in a flat plate shape parallel to the first flat plate portion,
wherein each of the connecting members run through a respective one of the connecting portions, and
wherein the first magnet, the abutting member, and the connecting portions are located on a same side of the first flat plate portion.

26. A medical table, comprising:
a table frame;
a table board movably arranged on the table frame; and
a braking mechanism comprising:
a first support configured to connect a first object;
a first magnet movably connected to the first support along a first straight line, the first magnet comprising one of an electromagnet or an electrically-controlled permanent magnet; and
a driving unit configured to drive the first magnet to move along the first straight line relative to the first support to enable the first magnet to reciprocate between a release position and a braking position,
wherein the first magnet in the braking position is configured to contact and magnetically attract a second object that is movable relative to the first object,
wherein the first support is connected to one of the table frame or the table board, and
wherein the other one of the table frame or the table board contacts the first magnet in the braking position and is configured to be magnetically attracted by a magnetic force of the first magnet.
